(19) United States
(12) Patent Application Publication
(10) Pub. No.: US 2022/0260489 A1
(43) Pub. Date: Aug. 18, 2022

(54) SYSTEM AND METHOD FOR ANALYZING MOLECULAR INTERACTIONS ON LIVING CELLS USING BIOSENSOR TECHNIQUES

(71) Applicant: Biosensing Instrument Inc., Tempe, AZ (US)

(72) Inventors: Nguyen Ly, Tempe, AZ (US); Tianwei Jing, Tempe, AZ (US)

(73) Assignee: Biosensing Instrument Inc., Tempe, AZ (US)

(21) Appl. No.: 17/736,389

(22) Filed: May 4, 2022

Related U.S. Application Data
(63) Continuation-in-part of application No. 17/074,274, filed on Oct. 19, 2020, now Pat. No. 11,327,014.
Application No. 17/074,274 is a continuation of application No. 16/423,733, filed on May 28, 2019, now Pat. No. 10,809,194.
Application No. 16/423,733 is a continuation of application No. PCT/US19/34087, filed on May 27, 2019.
(60) Provisional application No. 62/676,983, filed on May 27, 2018.

(51) Int. Cl. G01N 21/552

(52) U.S. Cl. CPC G01N 21/553; G01N 2021/5903

(57) ABSTRACT

A method for measuring molecular interactions on a plurality of regions of interest (ROIs) of a sensor surface of a biosensor device. The method can include receiving respective biosensor response data for each ROI of the plurality of ROIs. The method further can include determining a sample group and a reference group for the plurality of ROIs. The sample group can include sample group ROIs of the plurality of ROIs, and the reference group can include reference group ROIs of the plurality of ROIs. The method also can include generating one or more sample data distributions based on one or more respective sample group binding parameters for each of the sample group ROIs derived from the respective biosensor response data for the each of the sample group ROIs. The method further can include generating one or more reference data distributions based on one or more respective reference group binding parameters for each of the reference group ROIs derived from the respective biosensor response data for the each of the reference group ROIs. Other embodiments are disclosed.

### CROSS-REFERENCE TO RELATED APPLICATIONS

This application is a continuation-in-part of, and claims priority to U.S. patent application Ser. No. 17/074,274, filed Oct. 19, 2020. U.S. patent application Ser. No. 17/074,274 is a continuation of, and claims priority to U.S. patent application Ser. No. 16/423,733, filed May 28, 2019 and issued as U.S. Pat. No. 10,809,194 on Oct. 20, 2020. U.S. patent application Ser. No. 16/423,733 is a continuation of, and claims priority to PCT/US19/34087, filed May 27, 2019. PCT/US19/34087 claims priority to U.S. Provisional Patent Application No. 62/676,983, filed May 27, 2018. U.S. patent application Ser. No. 17/074,274, U.S. patent application Ser. No. 16/423,733, PCT/US19/34087, and U.S. Provisional Patent Application No. 62/676,983 are incorporated herein by reference in their entirety.

### TECHNICAL FIELD

This disclosure relates generally to biosensor systems, and methods to use such systems for measuring molecular interactions.

### BACKGROUND

Label-free detection via biosensor devices such as surface plasmon resonance instruments is a popular technique for monitoring molecular interactions in real-time. However, traditional biosensor devices or systems are not adequate for the study of heterogeneity effects naturally occurring in cell population because they either have limited fields of view or are not designed for imaging cellular structures or phenotypes that often have disordered patterns and structures. Therefore, a need exists for a system and a method configured to have a large field of view and a high resolution along with a rigorous algorithm for measuring molecular interactions on heterogeneous surfaces.

### BRIEF DESCRIPTION OF THE DRAWINGS

To facilitate further description of the embodiments, the following drawings are provided in which:

FIG. 1 illustrates a perspective view of a biosensor system, according to an embodiment;

FIG. 2 shows a side view of the biosensor system with the housing of an optical assembly cut open, according to the embodiment in FIG. 1;

FIG. 3 shows a perspective view of a biosensor system, according to an embodiment;

FIG. 4 shows an angle-response profile curve, according to an embodiment;

FIG. 5 shows a perspective view of a biosensor system including a sensor with multiple region of interests (ROIs), according to an embodiment;

FIG. 6 shows angle-response profile curves, according to an embodiment;

FIG. 7 shows angle-response profile curves, according to an embodiment;

FIG. 8 shows a partial angle-response profile curve with blocked regions marked with a letter N, according to an embodiment;

FIG. 9 shows angle-response profile curves for various ROIs, according to an embodiment;

FIG. 10 shows a histogram of ROI counts versus incident angles, according to an embodiment;

FIG. 11 illustrates an image of a sensor surface, with a grid superimposed on the image to divide the image into multiple ROIs, according to an embodiment;

FIG. 12 shows a data map of the binding activities on the sensor surface, according to the embodiment in FIG. 11;

FIG. 13 shows an image of the mapped binding activities on the sensor surface, according to the embodiment in FIGS. 11-12;

FIG. 14 shows histograms of the distributions of binding parameters, according to an embodiment;

FIG. 15 shows an Isoaffinity diagonal plot illustrating a relation of the distributions of the binding parameters, according to the embodiment in FIG. 14;

FIG. 16 illustrates an image of a sensor surface, with a grid superimposed on the image to divide the image into multiple ROIs, according to an embodiment;

FIG. 17 shows a data map of the binding activities on the sensor surface, according to the embodiment in FIG. 16;

FIG. 18 shows a histogram plot for the distribution of a binding parameter for ROIs, in a sample group (Group A), according to an embodiment;

FIG. 19 shows a histogram plot for the distribution of the binding parameter for ROIs in a reference group (Group B), according to the embodiment in FIG. 18;

FIG. 20 shows histogram plots for the distributions of a binding parameter for ROIs, in a sample group (Group A) or a reference group (Group B) respectively, according to another embodiment;

FIG. 21 shows histogram plots for the distributions of a binding parameter for ROIs, in a sample group (Group A) or a reference group (Group B) respectively, according to yet another embodiment;

FIG. 22 shows an image of a sensor surface, with a grid superimposed on the image to divide the image into multiple ROIs and exemplary regions marked, according to an embodiment;

FIG. 23 shows Total Internal Reflection (TIR) critical-angle-reflectivity profile curves, according to an embodiment;

FIG. 24 illustrates a flow chart for a method of measuring molecular interactions on a plurality of regions of interest (ROIs) of a sensor surface of a biosensor device;

FIG. 25 illustrates a front elevation view of a computer system that is suitable for implementing an embodiment of the method disclosed in FIG. 24; and

FIG. 26 illustrates a representative block diagram of an example of the elements included in the circuit boards inside a chassis of the computer system of FIG. 25.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, five seconds, ten seconds, thirty seconds, one minute, two minutes, or five minutes.

### Description of Examples of Embodiments

Various embodiments include a method for analyzing molecular interaction on cells. In some embodiments, the method can be performed via a device using optical imaging and surface plasmonic microscopy. The device can include a sensor surface on which cells can be attached, a light source, optical assemblies and optoelectronic detectors and imagers. The method can provide effective data processing and statistical data analysis of the binding behavior of molecular interaction on cells. In many embodiments, the methods and systems presented herein address one or more of the following: (a) the heterogeneity of multi-cell population in response to drug molecules, (b) how to mitigate local variations in sensor sensitivity (for example, magnitude of response versus surface plasmon resonance (SPR) angle changes) over the entire sensor surface and how to perform real-time measurements, and/or (c) the need to conduct statistically significant measurements using numerous regions of interest (ROIs) on a sensor.

Many embodiments can include a method for measuring molecular interactions on a plurality of regions of interest (ROIs) of a sensor surface of a biosensor device (e.g., an SPR sensor system). The method can be implemented via execution of computing instructions configured to run at one or more processors and stored at one or more non-transitory computer-readable media. In a number of embodiments, the method can include receiving respective biosensor response data for each ROI of the plurality of ROIs. The respective biosensor response data for the each ROI can include a respective biosensor response signal or a respective biosensor reflectivity signal for the each ROI measured by the biosensor device over a predetermined period of time (e.g., 10 seconds, 1 minute, 10 minutes, etc.). In some embodiments, the method further can include determining a sample group and a reference group for the plurality of ROIs. The sample group can include sample group ROIs of the plurality of ROIs, and each of the sample group ROIs can be supporting one or more samples for the molecular interactions to be measured. The reference group can include reference group ROIs of the plurality of ROI. The sample group ROIs can be absent from the reference group, while the reference group ROIs are absent from the sample group. In several embodiments, the method also can include generating one or more sample data distributions based on one or more respective binding parameters for each of the sample group ROIs derived from the respective biosensor response data for the each of the sample group ROIs. The method additionally can include generating one or more reference data distributions based on one or more respective binding parameters for each of the reference group ROIs derived from the respective biosensor response data for the each of the reference group ROIs.

A number of embodiments can include a system for measuring molecular interactions on a plurality of regions of interest (ROIs) of a sensor surface of a biosensor device. The system can include one or more processors, and one or more non-transitory computer-readable media storing computing instructions that, when executed on the one or more processors, cause the one or more processors to perform certain acts. In some embodiments, the acts can include receiving respective biosensor response data for each ROI of the plurality of ROIs. The respective biosensor response data for the each ROI can include a respective biosensor response signal or a respective biosensor reflectivity signal for the each ROI measured by the biosensor device over a predetermined period of time. The acts further can include determining a sample group and a reference group for the plurality of ROIs. The sample group can include sample group ROIs of the plurality of ROIs. Each of the sample group ROIs can be configured to support one or more samples for the molecular interactions to be measured. The reference group can include reference group ROIs of the plurality of ROIs. In many embodiments, the sample group ROIs are absent from the reference group, and the reference group ROIs are absent from the sample group. In some embodiments, the acts additionally can include generating one or more sample data distributions based on one or more respective binding parameters for each of the sample group ROIs derived from the respective biosensor response data for the each of the sample group ROIs. The acts further can include generating one or more reference data distributions based on one or more respective binding parameters for each of the reference group ROIs derived from the respective biosensor response data for the each of the reference group ROIs.

In various versions of the embodiments described in the previous two paragraphs, the biosensor device can comprise a surface plasmon resonance microscopy (SPRM) device; the biosensor response data can comprise SPR response data; the respective biosensor response signal can comprise a respective SPR response signal; and/or the respective biosensor reflectivity signal comprises a respective SPR reflectivity signal. In other variations of the embodiments described in the previous two paragraphs, the biosensor device can comprise a fluorescence device, a critical angle device, or the like.

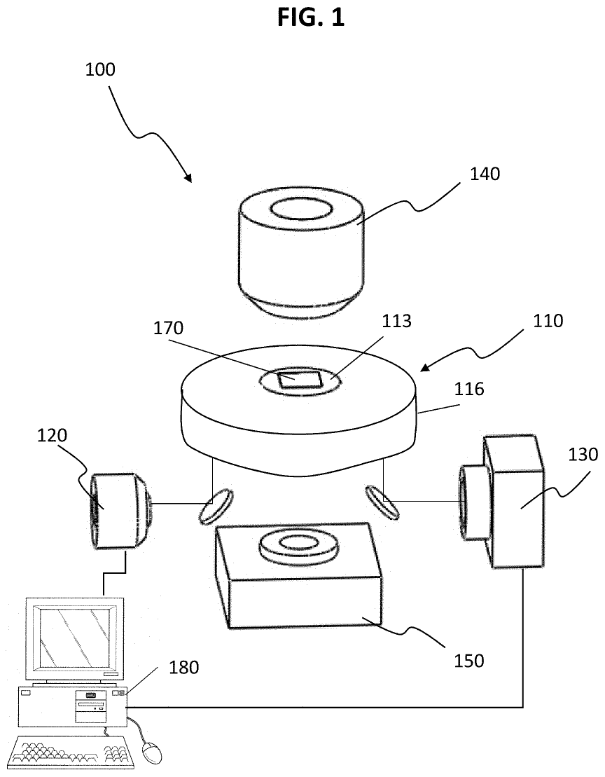

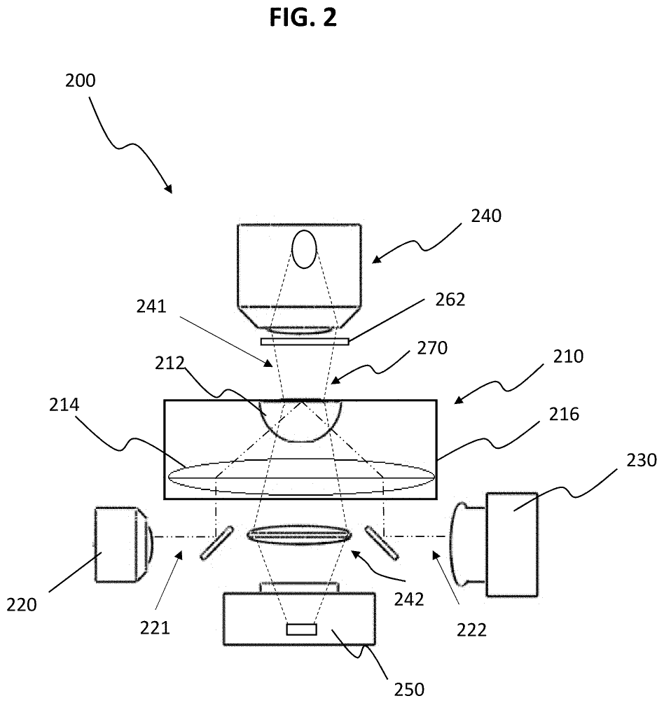

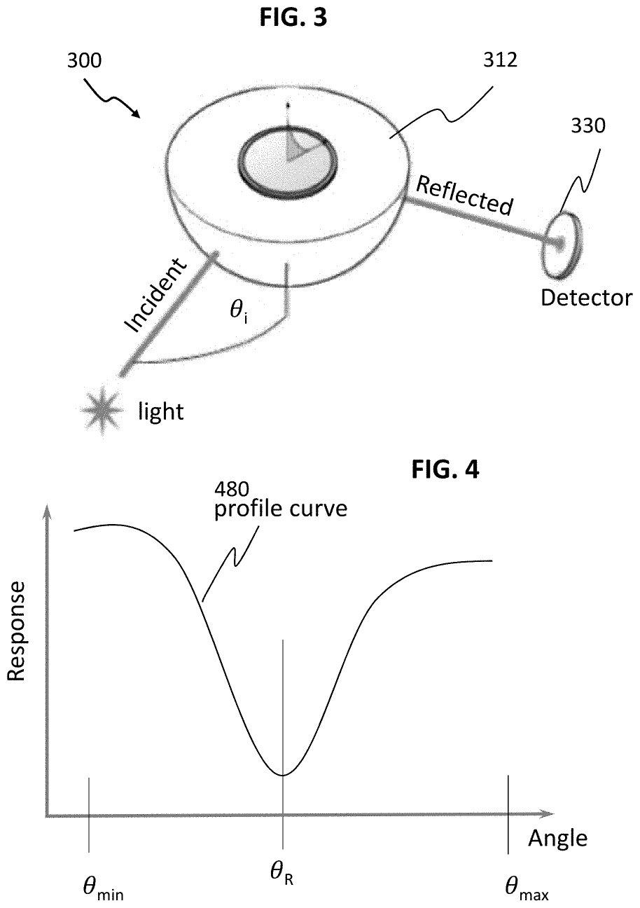

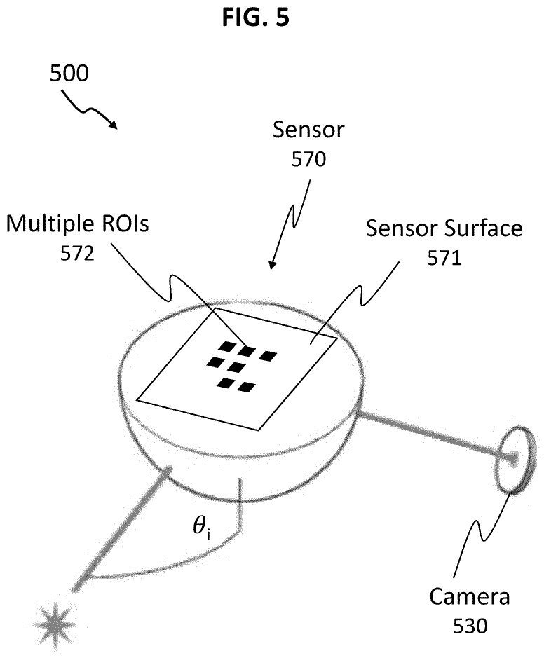

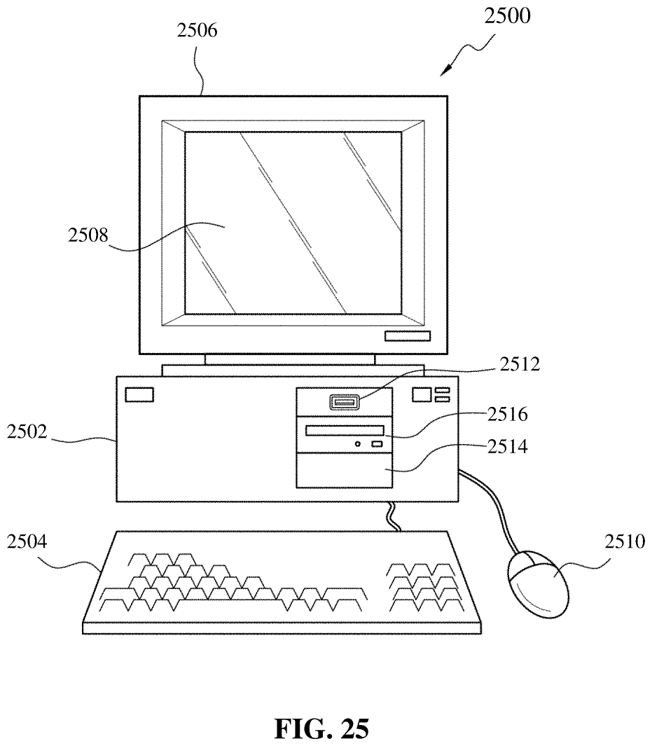

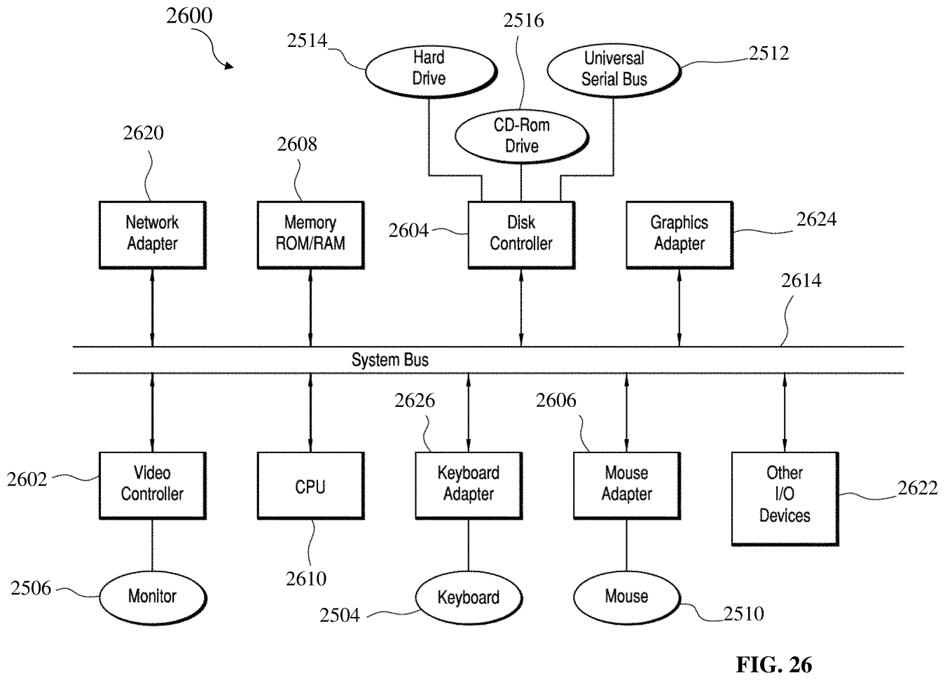

In a number of embodiments, a system (e.g., 100 (FIG. 1)) can include a computer (e.g., 180 (FIG. 1), 2500 (FIG. 25), or 2600 (FIG. 26)) and/or a SPRM device (e.g., 100 (FIG. 1), 200 (FIG. 2), 300 (FIG. 3), and/or 500 (FIG. 5)). In many embodiments, the SPRM device (see, e.g., 100 (FIGS. 1) and 200 (FIG. 2)) can have a large field of view and a high resolution. In some embodiments, the SPRM device can include: (a) an optical assembly (e.g., 110 (FIG. 1), or 210 (FIG. 2)), (b) an SPR light source (e.g., 120 (FIG. 1) or 220 (FIG. 2)) configured to emit a light beam for SPR imaging, and (c) an SPR camera (e.g., 130 (FIG. 1), 230 (FIG. 2), 330 (FIG. 3), or 530 (FIG. 5)) configured to capture an SPR image. The optical assembly can include a hemispherical prism (e.g., 212 (FIG. 2)) with a top surface configured to support an SPR sensor (e.g., 170 (FIG. 1) or 570 (FIG. 5)), and a high numerical aperture (NA) lens (e.g., 214 (FIG. 2)) (and/or a set of lenses) located distal from the top surface of the hemispherical prism. The SPR sensor can include a surface configured to contact a sample. The high NA lens (and/or the set of lenses) can be configured to refract the light beam toward the hemispherical prism. The high NA lens (and/or the set of lenses) and the hemispherical prism can be configured to condition (e.g., collimate and/or focus) the light beam toward the SPR sensor. The high NA lens (and/or the set of lenses) further can be configured to receive and refract the light beam toward the SPR camera, after the light beam is reflected by the surface of the SPR sensor.

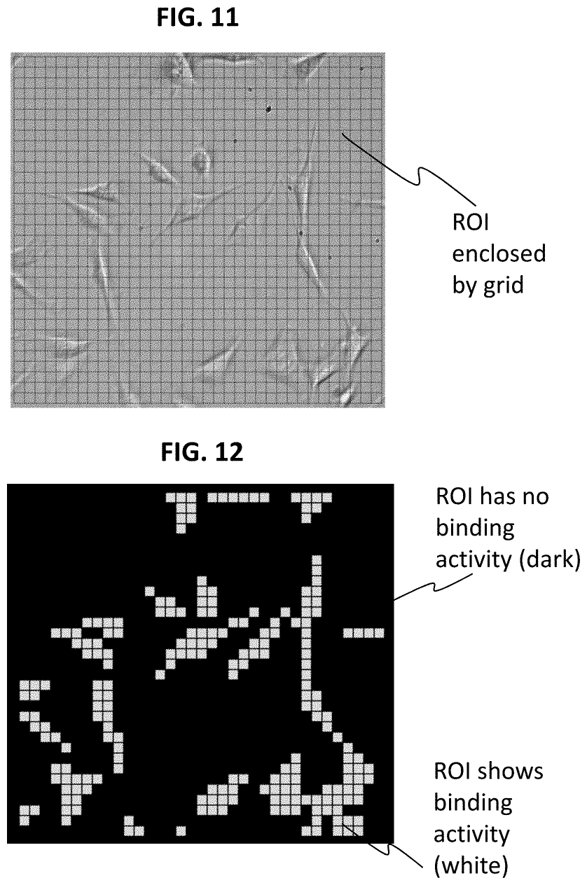

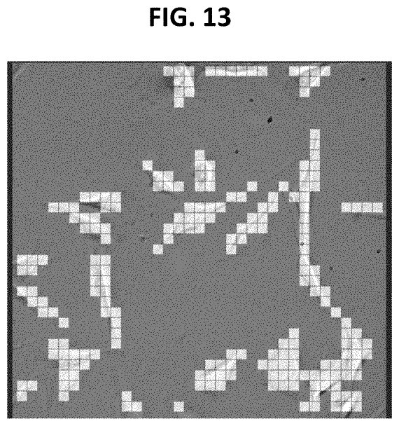

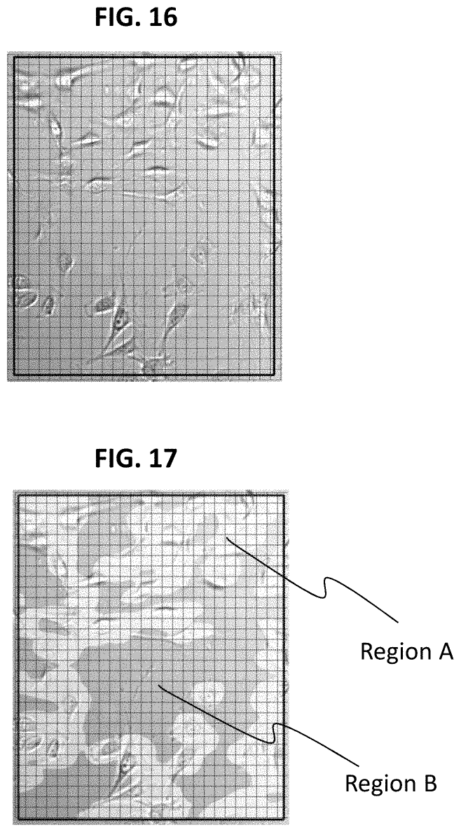

In some embodiments, the sensor surface (e.g., 571 (FIG. 5)) of the SPRM device can include a plurality of ROIs (see, e.g., FIGS. 11-13 & 16-17), and the SPRM device (e.g., 200 (FIG. 2), 300 (FIG. 3), and/or 500 (FIG. 5)) can be configured to measure the plurality of ROIs simultaneously. Each of the plurality of ROIs can be of any suitable size and/or dimension, such as a single pixel or 2×2 pixels in an SPR and/or optical images taken by the SPRM device. The ROIs can be fixed on the sensor surface or determined when or after an SPR or optical image of the sensor surface is taken. For example, the ROIs can be determined by superimposing a grid of a plurality of evenly distributed ROIs on an SPR image of the entire sensor surface (see, e.g., FIGS. 11, 16 & 17), and the SPR data (e.g., a SPR response or reflectivity signal) for each ROI (e.g., a pixel) enclosed by the grid can be measured, stored, and/or processed independently.

In some embodiments, the SPRM device (e.g., 200 (FIG. 2), 300 (FIG. 3), and/or 500 (FIG. 5)) can be configured to measure molecular interactions between drug molecules and protein receptors on cell membranes when an incident light beam is directed at a specific incident angle (θ₁) onto the sensor surface (e.g., 571 (FIG. 5)) to induce the surface plasmonic wave near its resonance. When an interaction happens, the refractive index near the sensor surface (e.g., 571 (FIG. 5)) can change locally and cause an SPR angle (θ_{R}) to change. The change in the SPR angle (Δθ_{R}) can be detected by a SPR detector camera (e.g., 130 (FIG. 1), 230 (FIG. 2), 330 (FIG. 3), and/or 430 (FIG. 4)) that is configured to receive the reflected light from the sensor (e.g., 170 (FIG. 1), 270 (FIG. 2), 370 (FIG. 3), and/or 570 (FIG. 5)). The SPR angle changes can be recorded with corresponding timestamps by the SPRM device and/or the computer. The data recorded can be used to derive the thermodynamic and kinetic behavior of the drug-cell interactions.

In several embodiments, the system (e.g., 100 (FIG. 1)) can be configured to: (a) receive SPR data for each ROI of the sensor surface (e.g., 571 (FIG. 5)) by: (i) retrieving the SPR data previously measured by the SPRM device (e.g., 200 (FIG. 2), 300 (FIG. 3), and/or 500 (FIG. 5)), or (ii) using the SPRM device to detect molecular interactions (e.g., molecular interactions between drug molecules and protein receptors on cell membranes) on the each ROI; and (b) use a computer (e.g., 180 (FIG. 1), 2500 (FIG. 25), or 2600 (FIG. 26)) to process and/or analyze the SPR data corresponding to the molecular interactions detected by the SPRM device (e.g., 200 (FIG. 2), 300 (FIG. 3), and/or 500 (FIG. 5)).

Because of the heterogeneity of the cell population on the sensor surface, in order to effectively measure these interactions, the sensor surface can be divided into various groups of ROIs. The optimal sensitivity of each ROI can be determined, and thus the data collected from each ROI can be processed to gain more meaningful information on the behavior of these interactions. In similar or different embodiments, the system (e.g., 100 (FIG. 1)) can include a biosensor device, in addition to or in lieu of the SPRM device, to detect the molecular interactions.

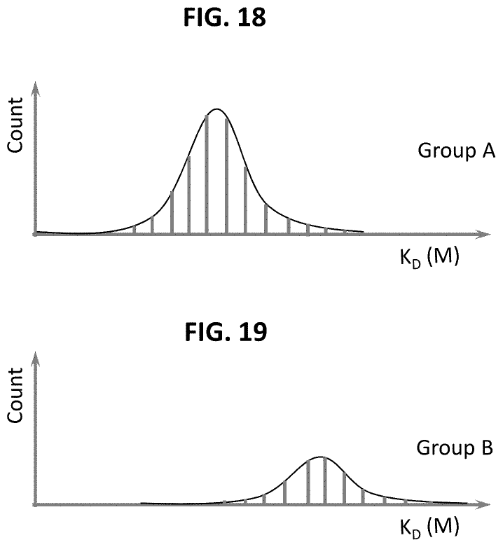

In several embodiments, the computer (e.g., 180 (FIG. 1), 1800 (FIG. 18), or 1900 (FIG. 19)) can be configured to control the SPRM device (e.g., 200 (FIG. 2), 300 (FIG. 3), and/or 400 (FIG. 5)). For example, the computer can be configured to cause the SPRM device to: (a) adjust the relative locations and/or orientations of one or more components (e.g., SPR light source 120 (FIG. 1), bright field light source 140 (FIG. 1), bright field camera 150 (FIG. 1), lens 214 (FIG. 2), prism 312 (FIG. 3), and/or SPR camera 530 (FIG. 5), etc.) of the SPRM device; (b) measure the sensor surface; (c) take SPR and/or optical images; (d) transfer in real-time the SPR data and/or the SPR/optical images to the computer, and/or (e) store the SPR data and/or SPR/optical images to a non-transitory computer-readable media and/or a remote database; and so forth.

In many embodiments, the sensor surface (e.g., 571 (FIG. 5)) can become heterogeneous when samples (e.g., molecules and/or cells) are immobilized on the sensor surface. The system (e.g., 100 (FIG. 1)) can be configured to prepare the SPR device (e.g., 200 (FIG. 2), 300 (FIG. 3), and/or 500 (FIG. 5)) before measuring the sensor surface (see, e.g., 1710 (FIG. 17)). In several embodiments, preparing the SPRM device before measuring can include determining a selected incident angle for the SPRM device to maximize the sensor sensitivity over the plurality of ROIs.

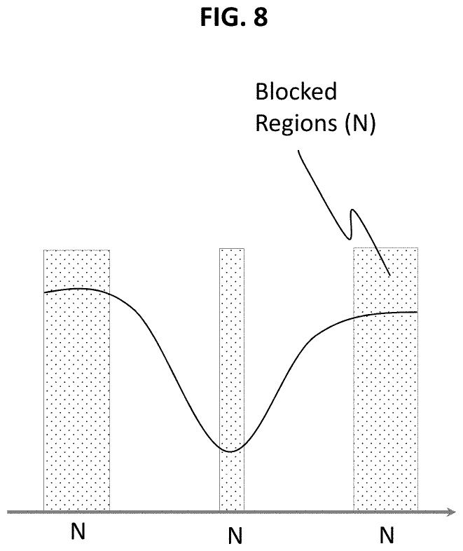

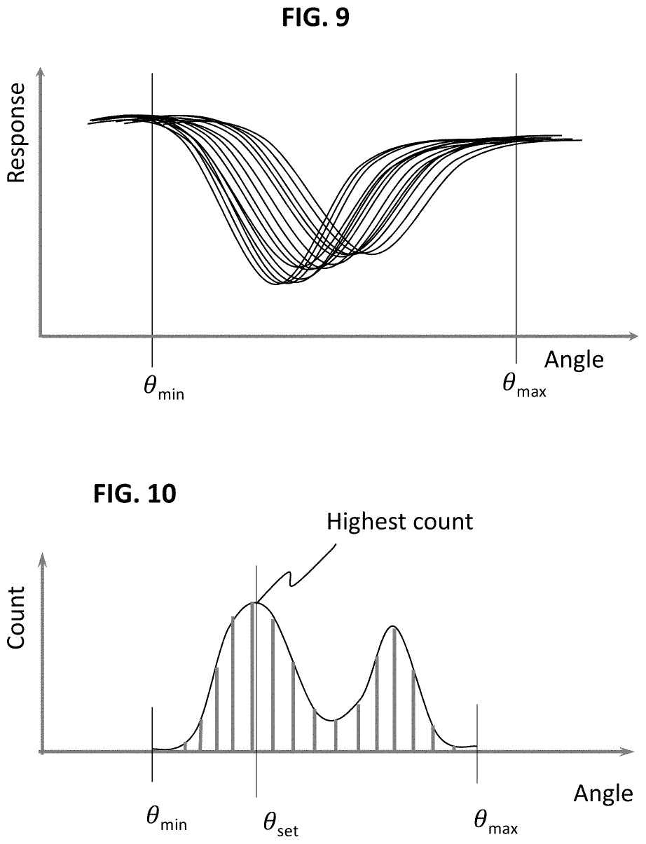

In a number of embodiments, determining the selected incident angle can include determining a respective ROI sensitivity for each ROI when the incident light is directed to the sensor surface at each incident angle of a predetermined range of incident angles (e.g., θᵢ (FIGS. 3-4) between θₘᵢₙ and θₘₐₓ). For example, the predetermined range of incident angles can be between 68 degrees (θₘᵢₙ) and 78 degrees (θₘₐₓ) for an optical/SPRM device (e.g., 200 (FIG. 2), 300 (FIG. 3), and/or 500 (FIG. 5)) that includes a 690-nm wavelength light source (e.g., 120 (FIG. 1) and/or 220 (FIG. 2)) and a SPR sensor (e.g., 170 (FIG. 1), 270 (FIG. 2), and/or 570 (FIG. 5)) with a sensor substrate of BK7 glass. The each incident angle (θᵢ) can be increased from θₘᵢₙ gradually until it reaches θₘₐₓ, such as 0=(68°+i*Δθ), where 0≤i≤1000 and Δθ=0.01. With the respective response of each ROI measured at each incident angle from θₘᵢₙ to θₘₐₓ, a respective SPR-angle-response profile curve for each ROI can be determined. The respective SPR-angle-response profile curve can include some regions (e.g., the blocked regions (N) in FIG. 8) where the slope of a tangent line of the profile curve is less than a threshold value (e.g., 0.035%/degree), indicating that the sensor sensitivity is low in those regions. Determining the selected incident angle further can include determining a respective ROI count for each incident angle in the predetermined range (e.g., FIG. 10), wherein the respective ROI count is the number of ROIs whose ROI sensitivity for the each incident angle is greater than, or equal to, a predetermined sensitivity threshold value (e.g., 0.035%/degree). Determining the selected incident angle additionally can include determining that the selected incident angle (e.g., θₛₑₜ (FIG. 10)) is one of the one or more incident angles for which the respective ROI counts are greatest among all of the incident angles within the predetermined range.

In a few embodiments, a standard calibration fluid (e.g., 90% phosphate-buffered saline (PBS) buffer) with a known SPR angle change (Δθ_{R}) (e.g., about 23 millidegrees for the 90% PBS buffer) can be used for determining the sensor sensitivity. For example, determining an ROI sensitivity for a ROI at an incident angle can include: (a) introducing a standard calibration fluid onto the sensor surface including the ROI, wherein the SPR angle change (Δθ_{R}) for the standard calibration fluid on the ROI at the incident angle is known; (b) measuring, by the SPRM device, a SPR response or reflectivity signal (R) for the ROI at the incident angle; and (c) determining the sensor sensitivity for the ROI at the incident angle by ΔR/Δθ_{R}. Any external perturbations that can produce any measurable biosensor responses (e.g., the calibration fluid used or the thermal change, etc.) also can be taken into account while determining the sensor sensitivity. The calculated ROI sensitivity for each ROI at each incident angle can be stored in a non-transitory computer-readable media and/or remote database. In certain embodiments, the known/predetermined SPR angle change (Δθ_{R}) also can be stored in the non-transitory computer-readable media and/or the remote database for future use.

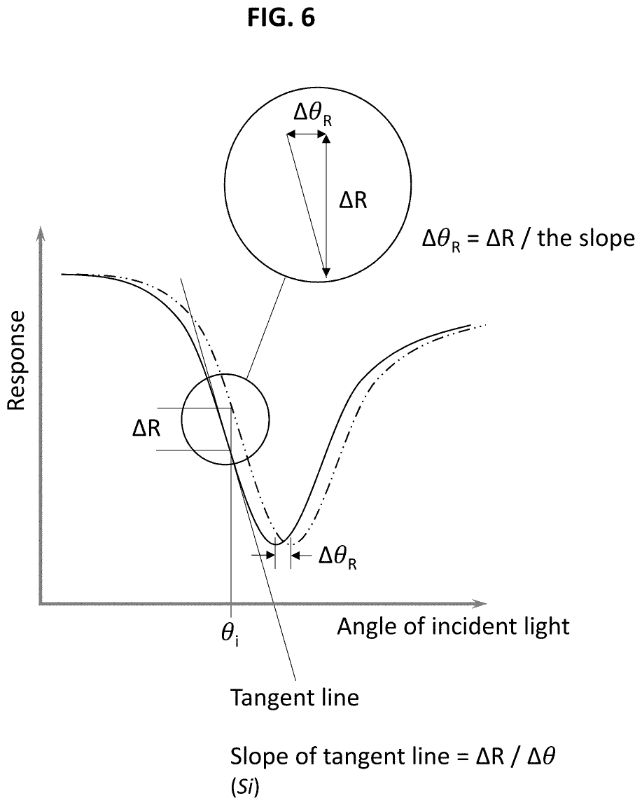

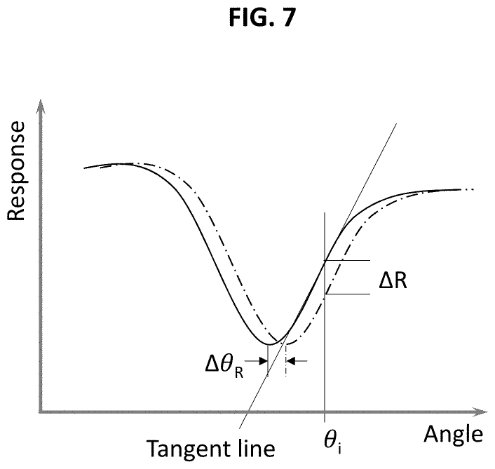

In some embodiments, determining an ROI sensitivity for an ROI (ROIᵢ) at an incident angle (θᵢ) (see, FIGS. 3-5)) can include: (a) obtaining a SPR-angle-response profile curve or critical-angle-reflectivity profile curve pre-measured for the ROI (see, FIG. 4, FIGS. 6-9, FIG. 23, etc.); (b) detecting, by the SPRM device, a SPR response or reflectivity signal (Rᵢ) for the ROI at the incident angle; (c) determining a SPR response or reflectivity signal change (ΔRᵢ) and a SPR angle change (Δθ_{Ri}) for the ROI (ROIᵢ) and the incident angle; and (d) determining the ROI sensitivity for the ROI (ROIᵢ) at the incident angle (θᵢ) based at least in part on the SPR angle change (Δθ_{Ri}) and the SPR response or reflectivity signal change (ΔRᵢ).

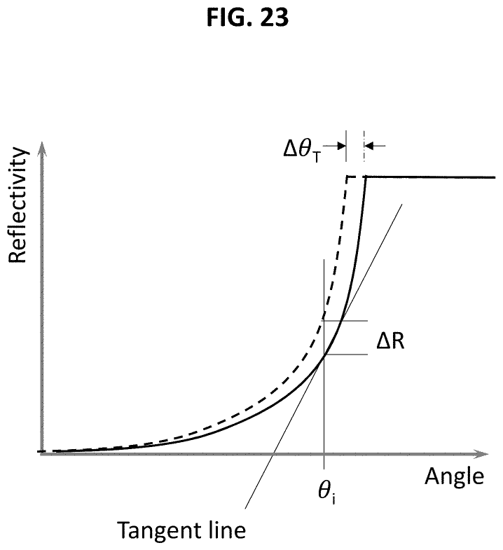

For a heterogeneous (non-uniform) sensor surface, the shape of the SPR-angle-response profile curve or critical-angle-reflectivity profile curve can vary at different ROI on the sensor surface, and the method can use a different SPR-angle-response profile curve or critical-angle-reflectivity profile curve for each ROI. If the SPR-angle-response profile curve (see, FIG. 4, FIGS. 6-9, etc.) or the critical-angle-reflectivity profile curve (see, FIG. 23) pre-measured for the ROI does not exist or is not accessible, the method can include obtaining the SPR-angle-response profile curve or critical-angle-reflectivity profile curve by: (a) measuring a SPR response or reflectivity signal (Rᵢ) for the ROI (ROIᵢ) with each incident angle (θᵢ) in a range (between θₘᵢₙ and θₘₐₓ); (b) plotting the SPR-angle-response profile curve or critical-angle-reflectivity profile curve for the ROI (ROIᵢ) based on the SPR response or reflectivity signal (Rᵢ); and/or (c) storing the SPR-angle-response profile curve or critical-angle-reflectivity profile curve for the ROI (ROIᵢ) to a non-transitory computer-readable media (e.g., 2512, 2514, or 2516 (FIG. 25)) and/or a remote database. If the pre-measured SPR-angle-response profile curve or critical-angle-reflectivity profile curve for the ROI exists, the pre-measured SPR-angle-response profile curve or critical-angle-reflectivity profile curve can be retrieved from the non-transitory computer-readable media and/or the remote database.

In a number of embodiments, the SPR response or reflectivity signal change (ΔRᵢ) and the SPR angle change (Δθ_{Ri}) for an ROI (ROIᵢ) at an incident angle (θᵢ) can be determined based on: (a) the SPR response or reflectivity signal (Rᵢ), measured by the SPRM device, for the ROI (ROIᵢ) at the incident angle (θᵢ); and/or (b) the SPR-angle-response profile curve or critical-angle-reflectivity for the ROI (ROIᵢ). For example, the SPR response or reflectivity signal change (ΔRᵢ) can be determined based on: (i) the SPR response or reflectivity signal (Rᵢ), and (ii) a pre-scanned SPR response value of the SPR-angle-response profile curve. In certain embodiments, the tangent line slope (Sᵢ) at the incident angle (θᵢ) of the SPR-angle-response profile curve (see, FIG. 4 and FIGS. 6-9) or the critical-angle-reflectivity profile curve (see, FIG. 23) for the ROI (ROIᵢ) can be used as the sensor sensitivity for the ROI (ROIᵢ) at the incident angle (θᵢ). Then, the SPR angle change (Δθ_{Ri}) can be determined by (Δθ_{Ri}). In several embodiments, a suitable higher-order curve fitting algorithm and/or a lookup table can be used to convert the SPR response or reflectivity signal change (ΔRᵢ) into the SPR angle change (Δθ_{Ri}) from the SPR-angle-response profile curve, and then the sensor sensitivity for the ROI (ROIᵢ) can be determined based on (ΔRᵢ/Δθ_{Ri}).

Experiments show that the aforementioned standard calibration fluid approach and/or the tangent line slope method can be used to produce a sufficiently accurate SPR angle change (Δθ_{Ri}) for small angle changes (e.g., Δθ_{R} <2 degrees), while the more complicated approach of a higher-order curve fitting algorithm and/or a lookup table can produce accurate results, even for large angle changes (e.g., Δθ_{R} >2 degrees). In many embodiments, the SPR response data for an ROI further can include any other information for or related to the ROI that has been calculated, obtained, and/or accumulated during the preparation of the SPRM device or the measuring by the SPRM device. For example, the SPR response data for an ROI can include, in addition to the SPR response or reflectivity signal, one or more binding parameters, the SPR-angle-response profile curve, the ROI sensitivity, the functional values of the curve fitting function, and/or an indication of the group the ROI is assigned to (e.g., a sample group, a reference group, and/or a group of ROIs that are removed from, or not assigned to, any of the sample group or the reference group(s)).

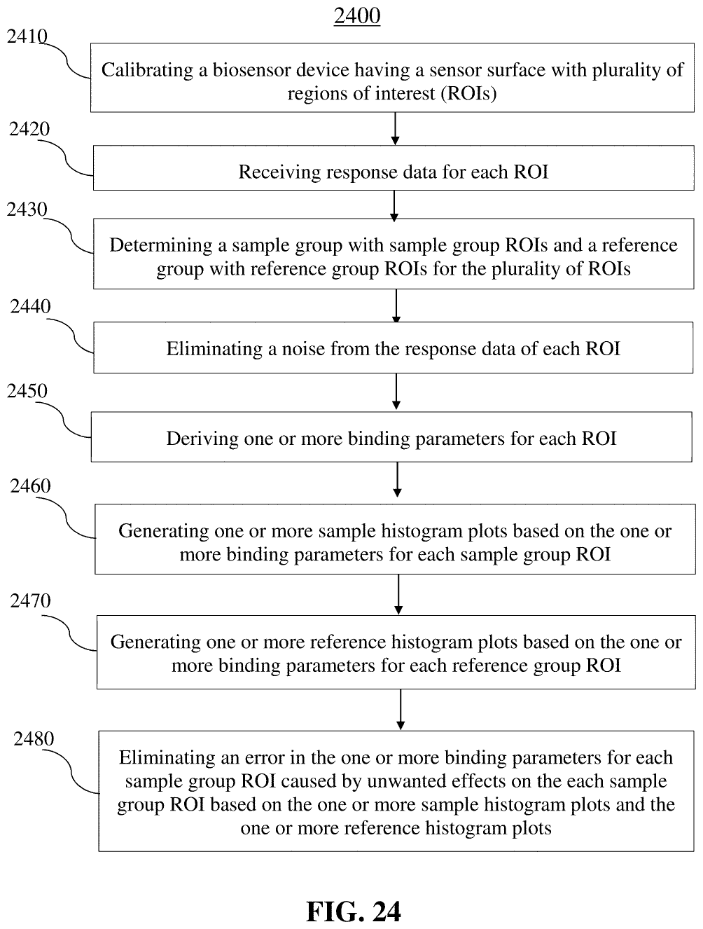

Various embodiments can include a method (e.g., 2400 (FIG. 24)) for measuring the molecular interactions on the plurality of ROIs of the sensor surface of the SPRM device (e.g., 200 (FIG. 2), 300 (FIG. 3), and/or 500 (FIG. 5)). In a number of embodiments, the method can be implemented via execution of computing instructions configured to run at one or more processors and stored at one or more non-transitory computer-readable media.

The method can comprise receiving respective SPR response data for each of the plurality of ROIs (e.g., 2420 (FIG. 24)). The respective SPR response data for each of the plurality of ROIs can include a respective SPR response or reflectivity signal for the each of the plurality of ROIs measured by the SPRM device over a predetermined period of time. Receiving respective SPR response data for each of the plurality of ROIs can include: (a) measuring, by the SPRM device in real-time, the respective SPR response or reflectivity signal for each of the plurality of ROIs over the predetermined period of time; and/or (b) retrieving the respective SPR response data for each of the plurality of ROIs from one or more non-transitory computer-readable media and/or a remote database. In many embodiments, measuring the respective SPR response or reflectivity signal for each of the plurality of ROIs can include superimposing a grid of the plurality of evenly distributed ROIs on an SPR image of the entire sensor surface (see, e.g., FIG. 11), and measuring the respective SPR response or reflectivity signal at each ROI enclosed by the grid. FIG. 12 shows an exemplary spatial data map of the binding activities (e.g., the ROIs in white squares) measured over the ROIs.

In some embodiments, the method can include automatically preparing the SPRM device (e.g., 2410 (FIG. 24)) as aforementioned, and after preparing the SPRM device, using the SPRM device to measure the plurality of ROIs to obtain the respective SPR response or reflectivity signal for each of the plurality of ROIs (see, e.g., 2420 (FIG. 24)). In similar or different embodiments, the method can include using a pre-prepared SPRM device to measure the plurality of ROIs to obtain the respective SPR response or reflectivity signal for each of the plurality of ROIs (see, e.g., 2420 (FIG. 24)).

In a number of embodiments, the method further can include determining a sample group and a reference group for the plurality of ROIs (e.g., 2430 (FIG. 24)). The sample group can include sample group ROIs of the plurality of ROIs. An ROI being in a sample group ROI can mean that the ROI is immobilized with one or more samples for the molecular interactions to be measured by the SPRM device. The reference group can comprise reference group ROIs of the plurality of ROIs. Each of the reference group ROIs of the reference group can be the same one of: (a) a bare area (e.g., the reference group ROI including no samples, such as cells), (b) having a modified surface on which samples cannot be immobilized or no SPR response can be detected, or (c) immobilized with alternative samples which have known binding behaviors, etc.

In some embodiments, the sample group and the reference group can be determined automatically, in real-time, by a computer (e.g., 180 (FIG. 1), 2500 (FIG. 25), or 2600 (FIG. 26)). In similar or different embodiments, the sample group and the reference group can be determined additionally or alternatively by a user using a manual technique. Determining the sample group and the reference group can include selecting the sample group ROIs based on characteristics (e.g., intensities) of the respective SPR response profile of each of the plurality of ROIs. For example, a sample group ROI can be selected when the intensity of the SPR response at the ROI is at least as great as a cut-off intensity (e.g., the average intensity or a predetermined value) of the SPR image at a particular angle.

In many embodiments, the sample group ROIs can be absent from the reference group, and the reference group ROIs can be absent from the sample group. In a few embodiments, the method can include determining one or more different reference groups. Each of the reference group ROIs of the same one of the one or more different reference groups can be the same one of: (a) a bare area, (b) including a modified surface, or (c) immobilized with alternative samples which have known binding behaviors, and so forth. The reference group ROIs of different ones of the one or more different reference groups can be the same or different in terms of the features (a)-(c).

In a number of embodiments, determining the sample group and the reference group for the plurality of ROIs (e.g., 1730 (FIG. 17)) can include: (a) identifying the one or more samples from an optical image of the sensor surface, taken by a camera (e.g., bright field camera 150 (FIG. 1)) of the SPRM device (e.g., 200 (FIG. 2), 300 (FIG. 3), and/or 500 (FIG. 5)); and (b) automatically mapping a respective location of each of the one or more samples to the sample group ROIs. Identifying the one or more samples can be implemented by any suitable object detection technologies, including neural network-based or non-neural approaches.

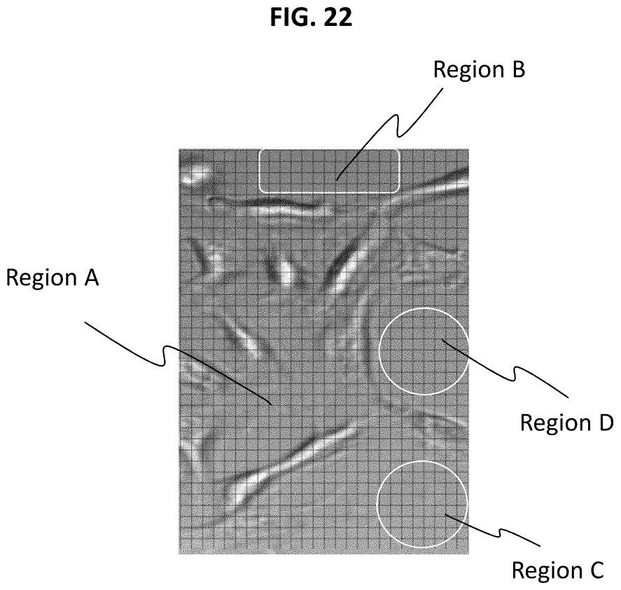

In some embodiments, determining the sample group and the reference group for the plurality of ROIs (e.g., 2430 (FIG. 24)) can include obtaining predetermined ROI grouping information. The predetermined ROI grouping information can comprise a predetermined mapping of sample group ROIs and reference group ROIs. For example, as shown in FIG. 22, a reference area comprising the reference group ROIs can be created by: (a) using a stamp to cover a small portion of the sensor surface and removing the stamp after cell attachment to leave a bare sensor surface, with no cell attached (see, Region B); (b) by using a spotting machine to modify a small area of the sensor surface (see, Region C); or (c) by placing a patch of alternative cells (see, Region D). These reference areas (e.g., Regions B, C, and D (FIG. 22)) can be designated as a negative control for some binding measurements. The predetermined ROI grouping information can be determined manually and/or automatically by a computer based on a respective ROI sensitivity for each ROI and/or biosensor response information at a predetermined incident angle for each ROI, etc.

In a number of embodiments, the method further can include eliminating a noise from the SPR response data of each ROI (see, e.g., 2440 (FIG. 24)). The noise to be eliminated can include, at least in part, the SPR response data of a representative reference group ROI. The representative reference group ROI can be an existing or virtual reference group ROI that is selected from the reference group ROIs by a user, determined by a computer, or predetermined based on the predetermined ROI grouping information. For instance, the computer (e.g., 180 (FIG. 1), 1800 (FIG. 18), etc.) can be configured to determine the representative reference group ROI randomly or when the SPR response data of the representative reference group ROI is the minimum, median, maximum, or any one among the reference group ROIs. In certain embodiments, every reference group ROI can be individually used as the representative reference group ROI to eliminate noise. For example, each reference group ROI can be subtracted from each sample group ROI to generate multiple data points for the each sample group ROI. In similar or alternate embodiments, one or more of the reference group ROIs that are locally significant for a sample ROI (e.g., one or more reference group ROIs located closest to the sample ROI) can form a sub-group to eliminate noise from the sample ROI. For example, the representative reference group ROI can be any of the average, sum, minimum, median, maximum, or any one of the sub-group of reference group ROIs. The sub-group of reference group ROIs for a sample ROI can overlap (e.g., having one or more common reference ROIs), entirely or partially, with the sub-group of reference group ROIs for another sample ROI. In some embodiments, the noise additionally or alternatively can be eliminated using one or more filters (e.g., a fast Fourier transform (FFT) filter and/or any other suitable digital filters).

In several embodiments, eliminating the noise from the SPR response data of each ROI (see, e.g., 2440 (FIG. 24)) further can include subtracting the SPR response data of the representative reference group ROI from the SPR response data of the each ROI (including the representative reference group ROI). After the subtracting, the method further can include determining a fit error for the SPR response data for the each ROI. For example, the fit error of the SPR response data (e.g., a binding parameter) for an ROI can be generated by any suitable comparative models, such as Chi-Squared, the standard deviation of residuals, etc. In a few embodiments, the method also can include removing a group ROI of the plurality of ROIs from a group (e.g., the sample group or the reference group) when the fit error for the group ROI, as normalized, is at least as great as a predetermined normalized cutoff error (e.g., 0.125, 0.130, 0.150, 0.160, etc.). The predetermined normalized cutoff error can be adjusted based on the quality of the SPR data, etc.

In some embodiments, the method also can include generating one or more sample distribution data based on one or more binding parameters for each sample group ROI (e.g., the one or more sample group binding parameters for each sample group ROI) (see, e.g., 2450 (FIG. 24)). The one or more binding parameters for each sample ROI can be derived from the respective SPR response data for the each sample group ROI. In several embodiments, the method additionally can include generating one or more reference data distributions based on one or more respective binding parameters for each of the reference group ROIs (e.g., the one or more reference group binding parameters for each reference group ROI) derived from the respective SPR response data for the each of the reference group ROIs (see, e.g., 2460 (FIG. 24)). In a number of embodiments, generating the one or more sample data distributions and the one or more reference data distributions (see, e.g., 2450 & 2460 (FIG. 24)) can be performed concurrently or in a different order.

The one or more binding parameters (e.g., the one or more respective sample or reference group binding parameters for each ROI) can include one or more kinetic parameters (e.g., an association rate constant (kₐ), a dissociation rate constant (k_{d}), etc.) and/or thermodynamic parameters (e.g., a binding affinity or equilibrium dissociation constant (K_{D})). The one or more binding parameters for an ROI can be determined by any suitable formula, e.g., a first order kinetic theory, based on the SPR angle change (Δθ_{R}) for the ROI detected by the SPRM device over the predetermined period of time.

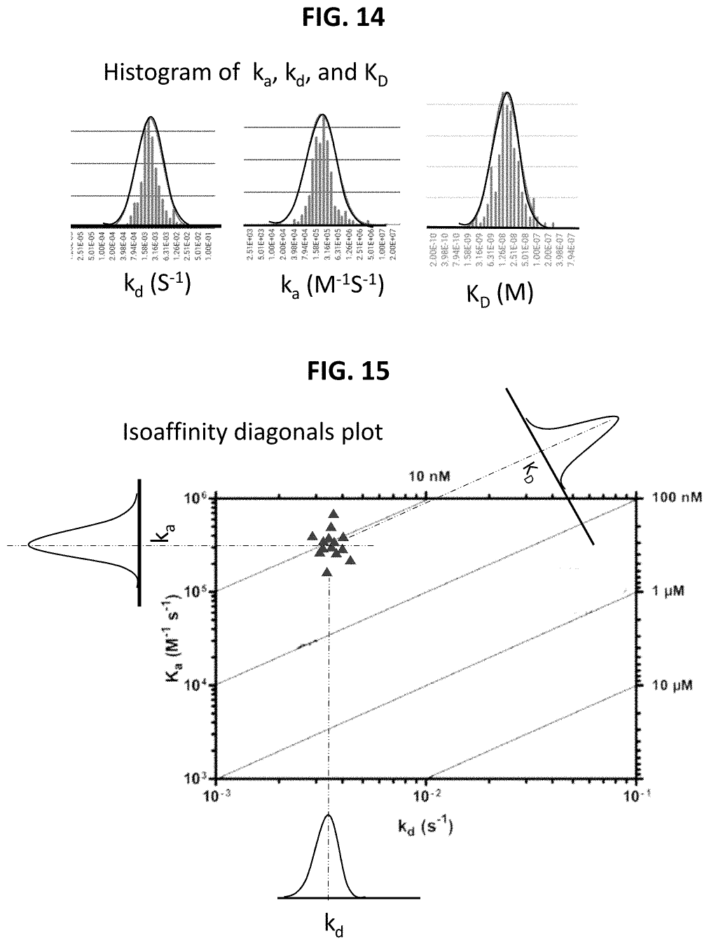

FIG. 14 shows some exemplary histograms of the distributions of measured binding parameters. Such histograms can be useful for statistical analysis of the binding parameters and for presenting a meaningful confidence interval on the measured data, demonstrating the number of active binding sites, and providing a measurement of cell heterogeneity. FIG. 15 illustrates the relation of the data distributions (e.g., histogram plots) for kₐ, k_{d}, and K_{D} with commonly used Isoaffinity diagonal plot to demonstrate the importance and validity of these data distributions in providing critical information for drug screening.

In a number of embodiments, the method further can include eliminating an error for each sample group ROI caused by one or more unwanted effects on the one or more sample data distributions and the one or more reference data distributions (see, e.g., 2470 (FIG. 24)). The one or more unwanted effects can include non-specific adsorption (NSA; also known as non-specific binding (NSB)), and/or secondary effects, etc., and the binding parameters for the reference group ROIs can be used to eliminate the error(s) in the binding parameters for the each sample group ROI, resulted from these unwanted effects. In some embodiments, eliminating the error(s) in the one or more binding parameters for each sample group ROI can include comparing the one or more sample data distributions (e.g., the histogram plots in FIGS. 18-21) and the one or more reference data distributions (e.g., the histogram plots in FIGS. 18-21) and/or subtracting the one or more reference data distributions from the one or more sample data distributions. In a few embodiments, prior to eliminating the error(s), the data distributions can be modified or transformed to make the comparison or evaluation of data distributions more meaningful. As an example, the transformation can be a mathematical normalization of count to the ratio of sample and reference areas.

For example, FIG. 18 shows a histogram plot in an embodiment that summarizes the distribution of the binding parameters (e.g., K_{D}) for the sample group ROIs with a fit error for each of the binding parameters in the histogram plot lower than a predetermined cutoff error (e.g., 0.1, 0.2, etc.). The histogram plot in FIG. 18 can represent valid biding activities for the sample group ROIs. In the same example, FIG. 19 illustrates another histogram plot showing the distribution of the binding parameters (e.g., K_{D}) for the reference group ROIs with a fit error for each of the binding parameters in this histogram plot less than the predetermined cutoff error. The histogram plot in FIG. 19 can be helpful for evaluating the extent of NSA and/or secondary effects that can cause the error(s) in the binding parameters (e.g., K_{D}) for the sample group ROIs.

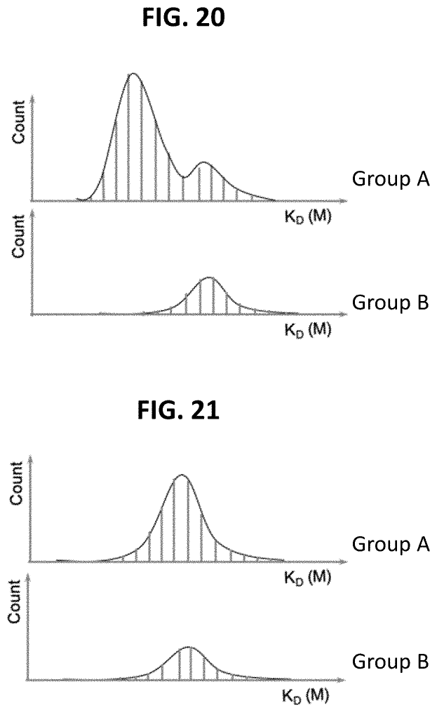

In another example in FIG. 20, a histogram for the sample group ROIs shows two or more peaks. An exemplary method can include comparing histogram plots between sample group ROIs (e.g., Group A) and reference group ROIs (e.g., Group B) and then removing the NSA and/or secondary effects as shown in FIG. 20 from the binding between drug molecules and cells. Further, histograms of similar peak values can also be compared with each other to show whether the observed responses are predominantly due to specific binding or non-specific binding.

In yet another example in FIG. 21, the two histogram plots between sample group ROIs (e.g., Group A) and reference group ROIs (e.g., Group B) show similar binding values. Instead of using histogram plots for the reference group ROIs (e.g., Group B) to remove NSA or secondary effects as aforementioned, the method can include using a different measurement buffer and/or different sensor treatment to create a different reference area (e.g., reference group ROIs) that has reduced NSA or secondary effects.

Various embodiments can include a system for measuring molecular interactions on a plurality of regions of interest (ROIs) of a sensor surface of a SPRM device. The system can include one or more processors and one or more non-transitory computer-readable media storing computing instructions that, when executed on the one or more processors, cause the one or more processors to perform acts. In many embodiments, the acts can include retrieving respective SPR response data for each ROI of the plurality of ROIs, wherein the respective SPR response data for the each ROI include a respective SPR response or reflectivity signal for the each ROI measured by the SPRM device over a predetermined period of time.

In a number of embodiments, the acts further can include determining a sample group and a reference group for the plurality of ROIs. The sample group can comprise sample group ROIs of the plurality of ROIs, and the each of the sample group ROIs can be supporting one or more samples for the molecular interactions to be measured. The reference group can comprise reference group ROIs of the plurality of ROIs, while the sample group ROIs are absent from the reference group, and the reference group ROIs are absent from the sample group.

In some embodiments, the acts additionally can include generating one or more sample data distributions based on one or more respective binding parameters for each of the sample group ROIs derived from the respective SPR response data for the each of the sample group ROIs. The acts also can include generating one or more reference data distributions based on one or more respective binding parameters for each of the reference group ROIs derived from the respective SPR response data for the each of the reference group ROIs. Moreover, the acts can include eliminating an error for each of the sample group ROIs caused by one or more unwanted effects on the one or more sample data distributions and the one or more reference data distributions.

Turning ahead in the drawings, FIG. 25 illustrates a computer 2500, all of which or a portion of which can be suitable for implementing an embodiment of at least a portion of computer 180 (FIG. 1) and/or performing an embodiment of one or more blocks of method 2400 (FIG. 24). Computer 2500 includes a chassis 2502 containing one or more circuit boards (not shown), a USB (universal serial bus) port 2512, a Compact Disc Read-Only Memory (CD-ROM) and/or Digital Video Disc (DVD) drive 2516, and a hard drive 2514. A representative block diagram of the elements included on the circuit boards inside chassis 2502 is shown in FIG. 26. A central processing unit (CPU) 2610 in FIG. 26 is coupled to a system bus 2614 in FIG. 26. In various embodiments, the architecture of CPU 2610 can be compliant with any of a variety of commercially distributed architecture families.

Continuing with FIG. 26, system bus 2614 also is coupled to memory 2608 that includes both read only memory (ROM) and random access memory (RAM). Non-volatile portions of memory storage unit 2608 or the ROM can be encoded with a boot code sequence suitable for restoring computer 2500 (FIG. 25) to a functional state after a system reset. In addition, memory 2408 can include microcode such as a Basic Input-Output System (BIOS). In some examples, the one or more memory storage units of the various embodiments disclosed herein can comprise memory storage unit 2608, a USB-equipped electronic device, such as, an external memory storage unit (not shown) coupled to universal serial bus (USB) port 2512 (FIGS. 25-26), hard drive 2514 (FIGS. 25-26), and/or CD-ROM or DVD drive 2516 (FIGS. 25-26). In the same or different examples, the one or more memory storage units of the various embodiments disclosed herein can comprise an operating system, which can be a software program that manages the hardware and software resources of a computer and/or a computer network. The operating system can perform basic tasks such as, for example, controlling and allocating memory, prioritizing the processing of instructions, controlling input and output devices, facilitating networking, and managing files. Some examples of common operating systems can comprise Microsoft® Windows® operating system (OS), Mac® OS, UNIX® OS, and Linux® OS.

As used herein, “processor” and/or “processing module” means any type of computational circuit, such as but not limited to a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, or any other type of processor or processing circuit capable of performing the desired functions. In some examples, the one or more processors of the various embodiments disclosed herein can comprise CPU 2610.

In the depicted embodiment of FIG. 26, various I/O devices such as a disk controller 2604, a graphics adapter 2624, a video controller 2502, a keyboard adapter 2526, a mouse adapter 2606, a network adapter 2620, and other I/O devices 2522 can be coupled to system bus 2514. Keyboard adapter 2526 and mouse adapter 2506 are coupled to a keyboard 2504 (FIGS. 25 and 26) and a mouse 2510 (FIGS. 25 and 26), respectively, of computer 2600 (FIG. 26). While graphics adapter 2624 and video controller 2602 are indicated as distinct units in FIG. 26, video controller 2602 can be integrated into graphics adapter 2624, or vice versa in other embodiments. Video controller 2602 is suitable for refreshing a monitor 2506 (FIGS. 25 and 26) to display images on a screen 2508 (FIG. 25) of computer 2500 (FIG. 25). Disk controller 2604 can control hard drive 2514 (FIGS. 25 and 26), USB port 2512 (FIGS. 25 and 26), and CD-ROM or DVD drive 2516 (FIGS. 25 and 26). In other embodiments, distinct units can be used to control each of these devices separately.

In some embodiments, network adapter 2620 can comprise and/or be implemented as a WNIC (wireless network interface controller) card (not shown) plugged or coupled to an expansion port (not shown) in computer system 2500 (FIG. 25). In other embodiments, the WNIC card can be a wireless network card built into computer system 2500 (FIG. 25). A wireless network adapter can be built into computer system 2500 (FIG. 25) by having wireless communication capabilities integrated into the motherboard chipset (not shown), or implemented via one or more dedicated wireless communication chips (not shown), connected through a PCI (peripheral component interconnector) or a PCI express bus of computer system 2500 (FIG. 25) or USB port 2512 (FIG. 25). In other embodiments, network adapter 2620 can comprise and/or be implemented as a wired network interface controller card (not shown).

Although many other components of computer 2500 (FIG. 25) are not shown, such components and their interconnection are well known to those of ordinary skill in the art. Accordingly, further details concerning the construction and composition of computer 2500 and the circuit boards inside chassis 2502 (FIG. 25) need not be discussed herein.

When computer 2500 in FIG. 25 is running, program instructions stored on a USB drive in USB port 2512, on a CD-ROM or DVD in CD-ROM and/or DVD drive 2516, on hard drive 2514, or in memory 2608 (FIG. 26) are executed by CPU 2610 (FIG. 26). A portion of the program instructions, stored on these devices, can be suitable for carrying out all or at least part of the techniques described herein. In various embodiments, computer 2600 can be reprogrammed with one or more modules, applications, and/or databases to convert a general purpose computer to a special purpose computer.

Although computer system 2500 is illustrated as a desktop computer in FIG. 25, there can be examples where computer system 2500 may take a different form factor while still having functional elements similar to those described for computer system 2500. In some embodiments, computer system 2500 may comprise a single computer, a single server, or a cluster or collection of computers or servers, or a cloud of computers or servers. Typically, a cluster or collection of servers can be used when the demand on computer system 2500 exceeds the reasonable capability of a single server or computer. In certain embodiments, computer system 2500 may comprise a portable computer, such as a laptop computer. In certain other embodiments, computer system 2500 may comprise a mobile device, such as a smartphone. For example, computer 180 (FIG. 1) can be a mobile device, such as a smartphone. In certain additional embodiments, computer system 2500 may comprise an embedded system.

In many embodiments, the techniques described herein can provide a practical application and several technological improvements. In many embodiments, using a uniformly distributed ROI grid can advantageously remove user bias of preferentially defining and selecting any ROI on the sensor surface, and allow a blind measurement for all ROIs to derive a distribution of binding parameters over the sensor surface. In some embodiments, measuring the binding activities on different ROIs based on different biosensor profile curves (e.g., biosensor-angle-response profile curves and/or critical-angle-reflectivity profile curves) improves the accuracy of the measured binding activities over a heterogeneous sensor surface, on which cells, living or fixed, can be attached. In embodiments where the sensor device for measuring molecular interactions includes a large field of view and a high resolution, the method further can be advantageous because with the greater quantity of the plurality of ROIs, the more local binding activities can be observed and measured, and the aforementioned benefits can be better achieved.

Although measuring binding interactions has been described with reference to specific embodiments, it will be understood by those skilled in the art that various changes may be made without departing from the spirit or scope of the disclosure. For example, the methods, systems, and/or algorithms described in the embodiments above can be applied to a biosensor profile curve obtained by using a clear glass surface sensor undergoing Total Internal Reflection (TIR) process. FIG. 23 shows a TIR critical-angle-reflectivity profile curve (with measured reflectivity vs incident angle of the light) that is similar to a SPR profile curve. The TIR angle shift (Δθ_{T}) can be the quotient of the reflectivity change (ΔR) divided by the sensor sensitivity, where sensor sensitivity is the slope of the TIR profile curve at the incident angle (θᵢ). Accordingly, the disclosure of embodiments is intended to be illustrative of the scope of the disclosure and is not intended to be limiting. It is intended that the scope of the disclosure shall be limited only to the extent required by the appended claims.

Replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A method for measuring molecular interactions on a plurality of regions of interest (ROIs) of a sensor surface of a biosensor device, the method being implemented via execution of computing instructions configured to run at one or more processors and stored at one or more non-transitory computer-readable media and comprising:
receiving respective biosensor response data for each ROI of the plurality of ROIs, wherein the respective biosensor response data for the each ROI include a respective biosensor response signal or a respective biosensor reflectivity signal for the each ROI measured by the biosensor device over a predetermined period of time;
determining a sample group and a reference group for the plurality of ROIs, wherein:
the sample group comprises sample group ROIs of the plurality of ROIs;
each of the sample group ROIs is supporting one or more samples for the molecular interactions to be measured;
the reference group comprises reference group ROIs of the plurality of ROIs;
the sample group ROIs are absent from the reference group; and
the reference group ROIs are absent from the sample group;
generating one or more sample data distributions based on one or more respective sample group binding parameters for each of the sample group ROIs derived from the respective biosensor response data for the each of the sample group ROIs; and
generating one or more reference data distributions based on one or more respective reference group binding parameters for each of the reference group ROIs derived from the respective biosensor response data for the each of the reference group ROIs.

2. The method of claim 1 further comprising:
preparing the biosensor device; and
after preparing the biosensor device, using the biosensor device to measure the plurality of ROIs to obtain the respective biosensor response signal or the respective biosensor reflectivity signal for each of the plurality of ROIs.

3. The method of claim 2, wherein preparing the biosensor device comprises:
determining a respective ROI sensitivity for the each ROI of the plurality of ROIs at each incident angle of a predetermined range of incident angles to the plurality of ROIs;
determining a respective ROI count at each incident angle of the predetermined range of incident angles, wherein the respective ROI count is a count of ROIs of the plurality of ROIs for which the respective ROI sensitivity at the each incident angle is no less than a predetermined sensitivity threshold; and
determining a selected incident angle of the predetermined range of incident angles for the biosensor device, wherein the respective ROI count at the selected incident angle is no less than another respective ROI count at any other incident angle of the predetermined range of incident angles.

4. The method of claim 3, wherein determining the respective ROI sensitivity for the each ROI of the plurality of ROIs at the each incident angle of the predetermined range of incident angles further comprises:
obtaining a respective biosensor profile curve for the each ROI of the plurality of ROIs;
detecting, by the biosensor device, the respective biosensor response signal or the respective biosensor reflectivity signal for the each ROI at the each incident angle;
determining a respective biosensor signal change and a respective biosensor angle change for the each ROI at the each incident angle based on: (a) the respective biosensor response signal or the respective biosensor reflectivity signal for the each ROI at the each incident angle, and (b) the respective biosensor profile curve for the each ROI; and
determining the respective ROI sensitivity for the each ROI at the each incident angle based at least in part on the respective biosensor angle change and the respective biosensor signal change.

5. The method of claim 4, wherein obtaining the respective biosensor profile curve for the each ROI of the plurality of ROIs further comprises:
(a) measuring the respective biosensor response signal or the respective biosensor reflectivity signal for the each ROI at the each incident angle of the predetermined range of incident angles; and
generating the respective biosensor profile curve for the each ROI based on the respective biosensor response signal or the respective biosensor reflectivity signal for the each ROI at the each incident angle; or
(b) retrieving the respective biosensor profile curve, pre-measured for the each ROI, from a non-transitory computer-readable media or a remote database.

6. The method of claim 4, wherein the respective ROI sensitivity for the each ROI of the plurality of ROIs at the each incident angle of the predetermined range of incident angles is determined by one of:
a respective slope of the respective biosensor profile curve at the each incident angle;
a respective function value of a curve fitting function for the each ROI based at least in part on the each incident angle, the respective biosensor signal change, and the respective biosensor angle change; or
a respective table value of a look-up table based at least in part on the respective biosensor signal change and the respective biosensor angle change.

7. The method of claim 3, wherein determining the respective ROI sensitivity for the each ROI of the plurality of ROIs at the each incident angle of the predetermined range of incident angles further comprises:
introducing a standard calibration fluid onto the each ROI, wherein a respective biosensor angle change for the standard calibration fluid on the each ROI at the each incident angle is predetermined;
detecting, by the biosensor device, the respective biosensor response signal or the respective biosensor reflectivity signal for the each ROI at the each incident angle; and
determining the respective ROI sensitivity for the each ROI at the each incident angle based on (a) the respective biosensor response signal or the respective biosensor reflectivity signal and (b) the respective biosensor angle change.

8. The method of claim 1, wherein determining the sample group and the reference group for the plurality of ROIs further comprises:
(a) identifying the one or more samples from an optical image of the sensor surface taken by the biosensor device; and
automatically mapping a respective location of each of the one or more samples to the sample group ROIs; or
(b) obtaining predetermined ROI grouping information, wherein the predetermined ROI grouping information comprises a predetermined mapping of sample group ROIs and reference group ROIs.

9. The method of claim 1 further comprising eliminating a noise from the respective biosensor response data of the each ROI of the plurality of ROIs based at least in part on the respective biosensor response data of a representative reference group ROI of the reference group ROIs.

10. The method of claim 9, wherein eliminating the noise from the respective biosensor response data of the each ROI of the plurality of ROIs further comprises:
subtracting the respective biosensor response data of the representative reference group ROI from the respective biosensor response data of the each ROI;
after subtracting, determining a respective fit error for each of the one or more respective sample group binding parameters and the one or more respective reference group binding parameters; and
removing a group ROI of the plurality of ROIs from a group of the sample group ROIs or the reference group ROIs when the respective fit error for the group ROI is at least as great as a predetermined cutoff error.

11. The method of claim 1 further comprising eliminating an error for each of the sample group ROIs caused by one or more unwanted effects based on the one or more sample data distributions and the one or more reference data distributions.

12. A system for measuring molecular interactions on a plurality of regions of interest (ROIs) of a sensor surface of a biosensor device, the system comprising:
one or more processors; and
one or more non-transitory computer-readable media storing computing instructions that, when executed on the one or more processors, cause the one or more processors to perform functions comprising:
receiving respective biosensor response data for each ROI of the plurality of ROIs, wherein the respective biosensor response data for the each ROI include a respective biosensor response signal or a respective biosensor reflectivity signal for the each ROI measured by the biosensor device over a predetermined period of time;
determining a sample group and a reference group for the plurality of ROIs, wherein:
the sample group comprises sample group ROIs of the plurality of ROIs;
each of the sample group ROIs is supporting one or more samples for the molecular interactions to be measured;
the reference group comprises reference group ROIs of the plurality of ROIs;
the sample group ROIs are absent from the reference group; and
the reference group ROIs are absent from the sample group;
generating one or more sample data distributions based on one or more respective sample group binding parameters for each of the sample group ROIs derived from the respective biosensor response data for the each of the sample group ROIs; and
generating one or more reference data distributions based on one or more respective reference group binding parameters for each of the reference group ROIs derived from the respective biosensor response data for the each of the reference group ROIs.

13. The system of claim 12, wherein the computing instructions, when executed on the one or more processors, cause the one or more processors to further perform additional functions comprising:
preparing the biosensor device; and
after preparing the biosensor device, using the biosensor device to measure the plurality of ROIs to obtain the respective biosensor response signal or the respective biosensor reflectivity signal for each of the plurality of ROIs.

14. The system of claim 13, wherein preparing the biosensor device comprises:
determining a respective ROI sensitivity for the each ROI of the plurality of ROIs at each incident angle of a predetermined range of incident angles to the plurality of ROIs;
determining a respective ROI count at each incident angle of the predetermined range of incident angles, wherein the respective ROI count is a count of ROIs of the plurality of ROIs for which the respective ROI sensitivity at the each incident angle is no less than a predetermined sensitivity threshold; and
determining a selected incident angle of the predetermined range of incident angles for the biosensor device, wherein the respective ROI count at the selected incident angle is no less than another respective ROI count at any other incident angle of the predetermined range of incident angles.

15. The system of claim 14, wherein determining the respective ROI sensitivity for the each ROI of the plurality of ROIs at the each incident angle of the predetermined range of incident angles further comprises:
obtaining a respective biosensor profile curve for the each ROI of the plurality of ROIs;
detecting, by the biosensor device, the respective biosensor response signal or the respective biosensor reflectivity signal for the each ROI at the each incident angle;
determining a respective biosensor signal change and a respective biosensor angle change for the each ROI at the each incident angle based on: (a) the respective biosensor response signal or the respective biosensor reflectivity signal for the each ROI at the each incident angle, and (b) the respective biosensor profile curve for the each ROI; and
determining the respective ROI sensitivity for the each ROI at the each incident angle based at least in part on the respective biosensor angle change and the respective biosensor signal change.

16. The system of claim 15, wherein obtaining the respective biosensor profile curve for the each ROI of the plurality of ROIs further comprises:
(a) measuring the respective biosensor response signal or the respective biosensor reflectivity signal for the each ROI at the each incident angle of the predetermined range of incident angles; and
generating the respective biosensor profile curve for the each ROI based on the respective biosensor response signal or the respective biosensor reflectivity signal for the each ROI at the each incident angle; or
(b) retrieving the respective biosensor profile curve, pre-measured for the each ROI, from a non-transitory computer-readable media or a remote database.

17. The system of claim 15, wherein the respective ROI sensitivity for the each ROI of the plurality of ROIs at the each incident angle of the predetermined range of incident angles is determined by one of:
a respective slope of the respective biosensor profile curve at the each incident angle;
a respective function value of a curve fitting function for the each ROI based at least in part on the each incident angle, the respective biosensor signal change, and the respective biosensor angle change; or
a respective table value of a look-up table based at least in part on the respective biosensor signal change and the respective biosensor angle change.

18. The system of claim 14, wherein determining the respective ROI sensitivity for the each ROI of the plurality of ROIs at the each incident angle of the predetermined range of incident angles further comprises:
introducing a standard calibration fluid onto the each ROI, wherein a respective biosensor angle change for the standard calibration fluid on the each ROI at the each incident angle is predetermined;
detecting, by the biosensor device, the respective biosensor response signal or the respective biosensor reflectivity signal for the each ROI at the each incident angle; and
determining the respective ROI sensitivity for the each ROI at the each incident angle based on (a) the respective biosensor response signal or the respective biosensor reflectivity signal and (b) the respective biosensor angle change.

19. The system of claim 12, wherein determining the sample group and the reference group for the plurality of ROIs further comprises:
(a) identifying the one or more samples from an optical image of the sensor surface taken by the biosensor device; and
automatically mapping a respective location of each of the one or more samples to the sample group ROIs; or
(b) obtaining predetermined ROI grouping information, wherein the predetermined ROI grouping information comprises a predetermined mapping of sample group ROIs and reference group ROIs.

20. The system of claim 12, wherein the computing instructions, when executed on the one or more processors, cause the one or more processors to further perform additional functions comprising eliminating a noise from the respective biosensor response data of the each ROI of the plurality of ROIs based at least in part on the respective biosensor response data of a representative reference group ROI of the reference group ROIs.

21. The system of claim 20, wherein eliminating the noise from the respective biosensor response data of the each ROI of the plurality of ROIs further comprises:
subtracting the respective biosensor response data of the representative reference group ROI from the respective biosensor response data of the each ROI;
after subtracting, determining a respective fit error for each of the one or more respective sample group binding parameters and the one or more respective reference group binding parameters; and
removing a group ROI of the plurality of ROIs from the sample group ROIs or the reference group ROIs when the respective fit error for the group ROI is at least as great as a predetermined cutoff error.

22. The system of claim 12, wherein the computing instructions, when executed on the one or more processors, cause the one or more processors to further perform additional functions comprising eliminating an error for each of the sample group ROIs caused by one or more unwanted effects based on the one or more sample data distributions and the one or more reference data distributions.
